(19) United States
(12) Patent Application Publication
(10) Pub. No.: US 2024/0082355 A1
(43) Pub. Date: Mar. 14, 2024

(54) LIQUID FORMULATION OF PROTEIN AND METHODS OF PREPARING THE SAME

(71) Applicant: Hanmi Pharm. Co., Ltd., Gyeonggi-do (KR)

(72) Inventors: Hyung Kyu LIM, Gyeonggi-do (KR); Sang Yun KIM, Gyeonggi-do (KR); Sung Hee HONG, Gyeonggi-do (KR)

(73) Assignee: Hanmi Pharm. Co., Ltd., Gyeonggi-do (KR)

(21) Appl. No.: 18/263,143

(22) PCT Filed: Jan. 26, 2022
(86) PCT No.: PCT/KR2022/001406

§ 371 Date: Jul. 27, 2023

(30) Foreign Application Priority Data
Jan. 27, 2021 (KR) 10-2021-0011802

(51) Int. Cl. A61K 38/19; A61K 9/00; A61K 9/08; A61K 47/02; A61K 47/12; A61K 47/26; A61P 7/00

(52) U.S. Cl. CPC A61K 38/193; A61K 9/0019; A61K 9/08; A61K 47/02; A61K 47/12; A61K 47/26; A61P 7/00

(57) ABSTRACT

Provided are a liquid formulation of protein and a method of preparing the same. According to a liquid formulation containing a high concentrate of eflapegrastim and a method of preparing the same, the liquid formulation may have excellent solubility and stability, may have a high concentration of protein, and may be injected in a patient-friendly manner due to reduced irritation/pain at the administration site or patient discomfort.

### TECHNICAL FIELD

The present invention relates to liquid formulations of protein and methods of preparing the same. This application claims the benefit of Korean Patent Application No. 10-2021-0011802, filed on Jan. 27, 2021, in the Korean Intellectual Property Office, the disclosure of which is incorporated herein in its entirety by reference.

### BACKGROUND ART

Granulocyte-colony stimulating factor (G-CSF), is a cytokine which stimulates the division and differentiation of bone marrow stem cells and leucocytes and promotes the division and differentiation of cells outside the bone marrow. G-CSF is a glycoprotein having a molecular weight of 18,000 to 19,000 Daltons and a dielectric point (μl) of 6.1 (a pI value ranging from 5.5 to 6.1 depending on the degree of glycosylation).

Recombinant DNA technologies established the molecular and genetic properties of G-CSF. Since the cloning of human G-CSF genes from cDNA libraries constructed with mRNA separated from CHU-2 cells and human bladder cancer cell line 5637, it has become possible to produce G-CSF from mammalian cells and prokaryotic cells.

In terms of the commercial feasibility and efficiency of pharmaceutical protein formulations including proteins such as G-CSF as described above, the stability of a formulation can be achieved by incorporating additional molecules into the formulation. The stability of proteins can be improved by incorporating excipients which interact with the protein in a solution to keep the protein stable, soluble, and non-aggregated. For example, salt compounds and other ionic species may be additives in protein formulations.

These additives help prevent denaturation of the protein by binding to the protein in a non-specific manner, thus increasing the thermal stability of the protein. Salt compounds (for example, NaCl, KCl) have been successfully used in commercially available insulin formulations to prevent aggregation and precipitation. Amino acids (for example, histidine and arginine) were revealed to reduce changes in the secondary structure of proteins when used as formulation additives. Other examples of commonly used additives include polyalcohols materials such as glycerol and sugar alcohols, and nonionic (for example, Tween, Pluronic) surfactants.

Pharmaceutical additives should be soluble and nontoxic, and should be used at specific concentrations which provide an effect of stabilizing specific therapeutic proteins. Since the effect of stabilizing additives are protein-dependent and concentration dependent, each additive to be used in a pharmaceutical formulation should be carefully tested so as not to cause instability or other adverse effects on the chemical or physical composition of the corresponding formulation. Ingredients used to stabilize proteins may cause problems with the stability of protein over time or with the stability of protein with respect to environmental changes during storage.

In addition, pharmaceutical protein formulations should be formulated at high concentrations to enhance therapeutic effects. High-concentration protein formulations may have a smaller volume dose and are more economical in packing and storage, and thus are therapeutically advantageous. However, with the development of high-concentration protein formulations, there are problems in terms of preparation, stability, and patient pain, among others. For example, generally the aggregation or insolubility of protein increases along with the increase of protein concentration in formulations (Shire, S. J. et al., J. Pharm. Sci., 93, 1390(2004)). Accordingly, high-protein formulations exhibit adverse effects which do not occur with low-protein formulations, such as aggregation of proteins in a non-natural form and the formation of particulates, which may also occur with the use of additives which provide advantageous effects in low-protein formulations. In addition, the high viscosity of high-concentration proteins may interfere with preparation processes that use filtration, and may cause pain or additional adverse effects in patients during injection, and thus may be less patient-friendly. Accordingly, pharmaceutical protein formulations are required to maintain the balance in ingredients and concentrations in order to improve protein stability, patient friendliness, and therapeutic requirements, while carefully limiting any adverse effects.

Therefore, in regard to a protein formulation containing a high concentration of non-natural protein having high aggregation potential, there is a need for the development of formulation which is useful for therapeutic use, advantageous in terms of solubility and stability, and patient-friendly.

### DISCLOSURE OF INVENTION

### Technical Problem

In one aspect, this disclosure provides a liquid formulation of protein including a high concentration of eflapegrastim and a buffer material.

In another aspect, this disclosure provides a method of preparing the liquid formulation.

In another aspect, this disclosure provides an article of manufacture, including the liquid formulation.

Additional aspects will be set forth in part in the description which follows and, in part, will be apparent from the description, or may be learned by practice of the presented embodiments of the disclosure.

### Solution to Problem

According to an aspect, there is provided an aqueous eflapegrastim formulation which is a liquid formulation comprising eflapegrastim and a buffer material, wherein a concentration of the eflapegrastim is about 6 mg/mL to about 150 mg/mL; a patient-friendly (PF) index of the liquid formulation, represented by Equation 1, is 10 or less,

Patient-friendly (PF) index=Osm(mOsm/kg)/100+MGF(N) [Equation 1]
wherein, in Equation 1, Osm indicates the osmolarity value of the liquid formulation, and MGF indicates a value of maximum gliding force when the liquid formulation is injected with a 29-gauge (29 G) syringe at a rate of 2.835 mm/s;

Osmolarity of the liquid formulation is about 100 mOsm/kg to about 1000 mOsm/kg;

Maximum gliding force (MGF) of the liquid formulation is 7 N or less when injected with a 29-gauge (29 G) syringe at a velocity of about 2.835 mm/s, or 10 N or less at a velocity of about 4.725 mm/s; and
the remaining rate of the eflapegrastim after storage at a temperature of 23° C. to 27° C. and relative humidity of about 55% to 65% is 95% or greater, as measured by reversed phase high-performance liquid chromatography (RP-HPLC) or size-exclusion high-performance liquid chromatography (SE-HPLC).

In some embodiments, the liquid formulation has a conductivity of 15 mS/cm or less. In some embodiments, the remaining rate of eflapegrastim is 98% or greater.

In some embodiments, the liquid formulation has a viscosity of 4 cP or less at a room temperature of 20° C. to 25° C.

In some embodiments, a concentration of the buffer material is about 5 mM to about 100 mM. In some embodiments, the buffer material is citric acid and/or citrate.

In some embodiments, the liquid eflapegrastim formulation further comprises a stabilizing agent. In some embodiments, the stabilizing agent comprises mannitol. In some embodiments, a concentration of the mannitol is about 1% to about 20% (w/v) of the liquid formulation.

In some embodiments, the liquid eflapegrastim formulation further comprises a surfactant. In some embodiments, the surfactant is a polysorbate-based non-ionic surfactant. In some embodiments, the polysorbate-based non-ionic surfactant is selected from the group consisting of Polysorbate 20, Polysorbate 40, Polysorbate 60, and Polysorbate 80. In some embodiments, a final concentration of the polysorbate-based non-ionic surfactant after the liquid formulation is concentrated is about 0.0001% to about 0.5% (w/v) of the total liquid formulation.

In some embodiments, the liquid formulation has a pH of about 4 to about 8.

In some embodiments, the liquid eflapegrastim formulation further comprises a tonicity modifier. In some embodiments, the tonicity modifier is sodium chloride. In some embodiments, a concentration of the tonicity modifier is about 5 mM to about 200 mM.

In some embodiments, the liquid formulation is pre-treated using a purification column. In some embodiments, the pre-treated liquid formulation is concentrated after buffer exchange with a buffer which does not contain a polysorbate-based non-ionic surfactant.

In another aspect, this disclosure provides a liquid eflapegrastim formulation comprising eflapegrastim, a buffer material, and a surfactant, wherein
a concentration of the eflapegrastim is about 11 mg/mL to about 66 mg/mL; a concentration of the buffer material is about 5 mM to about 100 mM; and
a concentration of the surfactant after the liquid formulation is concentrated is about 0.001% to about 5% (w/v) of the total liquid formulation, and a concentration of the surfactant after the liquid formulation is concentrated is about 0.001% to about 5% (w/v) of the total liquid formulation.

In some embodiments, the surfactant is a polysorbate-based non-ionic surfactant.

In some embodiments, the liquid formulation comprises:
about 11 mg/mL to about 66 mg/mL of the eflapegrastim; about 5 mM to about 100 mM of citric acid and/or citrate; and about 0.001% to about 5% (w/v) of a polysorbate-based non-ionic surfactant.

In some embodiments, the polysorbate-based non-ionic surfactant is selected from the group consisting of Polysorbate 20, Polysorbate 40, Polysorbate 60, and Polysorbate 80.

In some embodiments, the liquid formulation comprises:
about 11 mg/mL to about 66 mg/mL of the eflapegrastim; about 5 mM to about 100 mM of sodium citrate; about 0.001% to about 0.5% (w/v) of Polysorbate 80; about 1% to about 20% (w/v) of mannitol; and about 5 mM to about 200 mM of sodium chloride.

In some embodiments, the osmolarity of the liquid formulation is about 100 mOsm/kg to about 800 mOsm/kg. In some embodiments, the liquid formulation has a conductivity of 15 mS/cm or less.

According to another aspect, there is provided a method of preparing the liquid formulation.

According to another aspect, there is provided an article of manufacture including the liquid formulation.

In yet another aspect, this disclosure provides a method of preventing, alleviating, or treating neutropenia in a patient having compromised white blood cell production comprising administering to the patient a therapeutically effective amount of the liquid eflapegrastim formulation as described herein.

In some embodiments, the neutropenia is severe chronic neutropenia or febrile neutropenia.

In some embodiments, the liquid eflapegrastim formulation is administered after the patient is treated with adjuvant or neoadjuvant chemotherapy. In some embodiments, the liquid eflapegrastim formulation is administered between 1 and 5 days after the patient is treated with adjuvant or neoadjuvant chemotherapy. In some embodiments, wherein the adjuvant or neoadjuvant chemotherapy is a combination of docetaxel and cyclophosphamide.

In some embodiments, a second dose of the liquid eflapegrastim formulation is administered between 15 and 25 days after a first dose of the liquid eflapegrastim formulation is administered to the patient.

In some embodiments, the therapeutically effective amount is a unit dosage form selected from: 25 μg/kg, 50 μg/kg, 100 μg/kg, and 200 μg/kg.

In some embodiments, the therapeutically effective amount is 13.2 mg of the liquid eflapegrastim formulation in a 0.6 mL dosage volume.

In some embodiments, the method further comprises administering to the patient a therapeutically effective amount of a second agent. In some embodiments, the second agent is an anti-cancer agent.

In some embodiments, the liquid eflapegrastim formulation is administered to the patient within about 6 hours, about 5 hours, about 2 hours, about 1 hour of completion of chemotherapy.

### Advantageous Effects of Invention

According to aspects of the present disclosure, in a liquid formulation containing a high concentration of eflapegrastim and a method of preparing the same, the liquid formulation may have excellent solubility and stability, even while containing a high concentration of protein, and may reduce irritation/pain at an administration site or patient discomfort, and thus may be injected in a patient-friendly manner.

### BRIEF DESCRIPTION OF DRAWINGS

FIG. 1 shows changes in remaining rate of eflapegrastim according to concentration of a polysorbate-based non-ionic surfactant in liquid formulations according to embodiments, as results of confirmation by reversed phase high-performance liquid chromatograph (RP-HPLC).

FIG. 2 shows changes in remaining rate of eflapegrastim according to concentration of a polysorbate-based non-ionic surfactant in liquid formulations according to embodiments, as results of confirmation by size-exclusion high-performance liquid chromatograph (SE-HPLC).

### MODE FOR THE INVENTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

An aspect provides a liquid formulation including a high concentration of eflapegrastim and a buffer material.

Eflapegrastim

As used herein, the term “eflapegrastim” is the international nonproprietary name (INN) of a long-acting granulocyte-colony stimulating factor (G-CSF) conjugate containing a recombinant human granulocyte-colony stimulating factor (hG-CSF) variants (see WHO Drug Information Volume 29, 2015). The eflapegrastim may be a conjugate of a bioactive peptide, a granulocyte-colony stimulating factor (G-CSF), a biodegradable polymer, and an immunoglobulin Fc region.

In addition, the immunoglobulin Fc used herein may be a human immunoglobulin Fc, or have a sequence of a closely related analogue thereof, for example, of animal origin, such as from cows, goats, pigs, mice, rabbits, hamsters, rats, or guinea pigs. The immunoglobulin Fc region may be an Fc region derived from IgG, IgA, IgD, IgE, or IgM, a combination thereof, or a hybrid thereof. For example, the immunoglobulin Fc region may be derived from IgG or IgM, which is most abundant in human blood, and more specifically, derived from IgG, which is known to improve the half-life of ligand binding proteins. The immunoglobulin Fc may be prepared by treating native IgG with a specific protease, or may be prepared from transformed cells using recombinant techniques. For example, the immunoglobulin Fc may be a recombinant human immunoglobulin Fc prepared from an E. coli transformant.

IgG may also be classified into IgG1, IgG2, IgG3, and IgG4 subclasses, and a combination or hybrid of these subclasses may also be available in the present invention. In particular, IgG may be IgG2 and IgG4 subclasses, and more particularly, may be the Fc region of IgG4 nearly free of effector function such as complement-dependent cytotoxicity (CDC). That is, the immunoglobulin Fc region for drug carriers herein may be a non-glycosylated Fc region derived from human IgG4. The human-derived Fc region is preferred to non-human-derived Fc regions which can cause undesirable immune responses, such as causing new antibodies to be generated by acting as antigens in the human body.

The eflapegrastim used herein may be prepared by conjugating the hG-CSF variant and the immunoglobulin Fc region. In this case, as a method of conjugation, the hG-CSF variant and the immunoglobulin Fc region may be cross-linked using a non-peptidyl polymer, or a fusion protein in which the hG-CSF variant and the immunoglobulin Fc region are linked may be prepared using recombinant technology. The non-peptidyl polymer used in cross-linking may be selected from the group consisting of biodegradable polymers such as polyethylene glycol, polypropylene glycol, a copolymer of ethylene glycol and propylene glycol, polyoxyethylated polyol, polyvinyl alcohol, polysaccharides, dextrans, polyvinyl ethyl ether, polylactic acid (PLA), and polylactic-glycolic acid (PLGA), lipid polymers, chitin, hyaluronic acid, and any combinations thereof. Any derivatives of these materials which are known in the art and derivatives which can be easily prepared at the level of skills in the art are all incorporated herein.

The hG-CSF variant herein may be extracted from mammals or may be chemically synthesized. The hG-CSF variant may also be obtained from the prokaryotic or eukaryotic organisms transformed with DNA which encodes the hG-CSF variant, using genetic recombination techniques, wherein colon bacteria (for example, E. coli), yeasts (for example, S. cerevisiae), or mammalian cells (for example, Chinese hamster ovary cells, monkey cells, and so forth) may be used as hosts. Depending on the host used, the hG-CSF variant expression product may be glycosylated with mammal or other eukaryotic carbohydrates, or may be non-glycoslated. When expressed in prokaryotic organisms, the hG-CSF variant expression product may include an initial methionine residue (Position-1). The hG-CSF variant suitable in the present invention may be a hG-CSF variant prepared with E. coli as the host cell.

In one embodiment, the eflapegrastim may include a recombinant human granulocyte-colony stimulating factor derivative^{17,65} Ser-G-CSF in which the 17^{th} cystein and 65^{th} proline residues of the native G-CSF are substituted with serine and the 1^{st} threonine is deleted. As described above, the non-native protein of eflapegrastim can cause aggregation of additional proteins and adverse effects, as compared with native proteins or ¹⁷Ser-G-CSF. Protein aggregation is a common problem in protein solutions and leads to an increase in the concentration and viscosity of proteins. The present disclosure provides a means to achieve a high-concentration, low-aggregation protein formulation. The formulation according to the present disclosure may have a stably high concentration of protein in a solution, and is advantageous for therapeutic purposes.

Meanwhile, the function and physiological activity of a protein such as a polypeptide are determined by the stereostructure of the protein, and the protein cannot exhibit an original specific function if a portion of the stererostructure that is associated with the function is changed. For example, it is known the fact that, despite a change in only a single amino acid sequence, when the amino acid corresponds to a functional site of the stereostructure of a protein and changes the stereostructure of the site, the function of the protein is affected. In addition, in the field of pharmaceutical formulations, the most common issue with protein and peptide formulations is the physicochemical stability of drugs, and in practice, characteristics of drugs are crucial in determining appropriate formulations in terms of successful delivery and stability. The first step in the development of protein drug formulations involves complete characterization of drug characteristics and stability in different formulations, and begins with consideration of physiochemical characteristics of a protein, such as isoelectric point, molecular weight, and total amino acid composition, by a person skilled in the art to which the present invention belongs (Jeffrery L. et al., 1994). That is, even with a change in only a single amino acid sequence, natural form proteins, as well as different protein drugs (for example, IL-1β), exhibit different physicochemical characteristics from those of natural form proteins, and thus, a unique solution to an approach in terms of stability of protein stabilization formulations is needed.

The liquid formulation according to embodiments is characterized by a higher stability even in a high-concentration condition of eflapegrastim including^{17,65} Ser-G-CSF with an additional amino acid modification, compared to a natural form hG-CSF or ¹⁷Ser-G-CSF.

The term “high concentration” used herein means a dose which enhances the advantageous effects of therapeutic use. For example, the eflapegrastim may be included in the formulation at a high concentration of about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/m. For example, the eflapegrastim may be included in the formulation at a high concentration of about 6 mg/mL to about 150 mg/mL, about 10 mg/mL to about 150 mg/mL, about 11 mg/mL to about 150 mg/mL, about 10 mg/mL to about 100 mg/mL, about 11 mg/mL to about 100 mg/mL, about 10 mg/mL to about 80 mg/mL, about 11 mg/mL to about 70 mg/mL, about 12 mg/mL to about 70 mg/mL, about 14 mg/mL to about 70 mg/mL, about 11 mg/mL to about 66 mg/mL, about 12 mg/mL to about 66 mg/mL, about 13 mg/mL to about 66 mg/mL, about 14 mg/mL to about 66 mg/mL, about 15 mg/mL to about 66 mg/mL, about 16 mg/mL to about 66 mg/mL, about 17 mg/mL to about 66 mg/mL, about 18 mg/mL to about 66 mg/mL, about 19 mg/mL to about 66 mg/mL, or about 20 mg/mL to about 66 mg/mL.

The expression “stable” herein means that the physical stability and/or chemical stability and/or biological stability of a protein are substantially retained during storage. Typically, a formulation is understood to be stable when a loss of the active ingredient of the formulation under specific storage conditions for a certain period of time is less than a certain level, for example, less than 10%, less than 7%, less than 5%, less than 4%, or less than 3%.

Concentration and Remaining Rate of Eflapegrastim

As described above, an increase in concentration of the eflapegrastim can adversely affect the remaining rate of the eflapegrastim. There may be unexpected impacts on the remaining rate of the eflapegrastim in terms of additional mutants relative to native hG-CSF. Accordingly, stability of the liquid formulation can be evaluated by, as a major factor, the aggregation of the protein drug. Protein drugs can form aggregates under shear stress or other physical or chemical environments, and such formation of aggregates is a factor which affects bioavailability reduction such as efficacy reduction, and thus is a major factor considered in the development of liquid formulations.

In one or more embodiments, the liquid formulation may have, after 4-week storage test, a protein remaining rate (for example, eflapegrastim) of about 95% or greater, about 96% or greater, about 97% or greater, or about 98% or greater, as measured by reversed-phase high-performance liquid chromatography (RP-HPLC) or size exclusion high-performance liquid chromatography (SE-HPLC) at a temperature of 23° C. to 27° C. and a relative humidity (RH) of 55% to 65%.

The remaining rate indicates a relative ratio of purity at a specific point of time to initial protein purity, and an nth-week remaining rate of protein (for example, eflapegrastim) in the liquid formulation may be defined by Equation 2.

n^{th}-week remaining rate(%)=n^{th}-week purity value/Initial purity value×100 [Equation 2]

In one or more embodiments, the RP-HPLC measurement may be carried out using a column suitable for a liquid formulation sample (for example, a C4 column (particle size: 5 μm, Interior diameter×length: 4.6 mm×250 mm)) at a temperature of about 40° C. to 80° C. The HPLC conditions may be summarized as follows: an eluent linear gradient system having a flow rate of 0.5 mL/min to 2.0 mL/min (for example, 1.0 mL/min); a mobile phase A including 0.05% to 1.0% of trifluoroacetic acid (for example, 0.1%) and 10% to 40% of acetonitrile (for example, 20%); and a mobile phase B including 0.05% to 1.0% of trifluoroacetic acid (for example, 0.1%) and 60% to 95% of acetonitrile (for example, 80%). In addition, the detector may be set to 214 nm.

In one or more embodiments, the SE-HPLC measurement may be carried out using a column suitable for a liquid formulation sample (for example, Protein LW-803 column (particle size: 5 μm, Ineterior Diameter×Length: 8.0 mm×300 mm)). The HPLC conditions may be summarized as follows: an isocratic gradient system having a flow rate of 0.3 mL/min to 1.2 mL/min (for example, 0.6 mL/min), a mobile phase including 10 mM of sodium phosphate, 50 mM to 300 mM sodium chloride, or 1% to 15% isopropyl alcohol. In addition, the detector may be set to 214 nm.

For example, the liquid formulation according to the present disclosure may maintain at least 95%, at least 96%, at least 97%, or at least 98% of the initial purity of the protein drug in monomeric form without producing aggregates or decomposition products under the above-described conditions. In other words, in the liquid formulation according to the present disclosure, about 5% or less, for example, about 4% or less, or about 3% or less of the initial content of the protein drug is converted into aggregates or decomposition products under the above-described conditions.

In general, such formulations are understood to have good stability when the remaining rate of the protein (for example, eflapegrastim) is maintained at about 95% after a 4-week storage test under accelerated conditions (25±2° C./60±5% RH). Such formulations are understood to have very good stability when the remaining rate of the protein is maintained at about 97% or greater after a 4-week storage test under accelerated conditions (25±2° C./60±5% RH). Such formulations are also understood to have excellent stability when the remaining rate of the protein is maintained at about 98% or greater after a 4-week storage test under the accelerated conditions (25±2° C./60±5% RH).

While not wishing to be bound by theory, as the liquid formulation according to the disclosure contains a high concentration of eflapegrastim, the remaining rate of eflapegrastim after long-term storage may be increased due to interactions between proteins. In addition, a specific amino acid sequence of the hG-CSF variant may also affect the increase in the remaining rate, which is considered due to the interaction between amino acids arising from the electrostatic bonding or chemical structure of charged amino acids.

For stability of the formulation, in addition to the eflapegrastim and the buffer material, other ingredients or materials known in the art may optionally be further included in the liquid formulation according to the present disclosure within the range not damaging the effects of the liquid formulation.

Stabilizing Agent

In one embodiment, the liquid formulation may include a stabilizing agent.

The term “stabilizing agent” may mean an excipient which improves or enhances stability. Examples of the stabilizing agent include mannitol, sorbitol, dextrose, trehalose, sucrose, raffinose, maltose, benzyl alcohol, biotin, bisulfite compounds, boron compounds, butylated hydroxyanisole (BHA), butylated hydroxytoluene (BHT), ascorbic acid and esters thereof, carotenoids, calcium citrate, acetyl-L-carnitine, chelating agents, chondroitin, chromium, citric acid, coenzyme Q-10, cysteine, cysteine hydrochloride, 3-dehydroshchimic acid (DHS), EDTA (ethylenediaminetetraacetic acid, edetate disodium), vitamin A and esters thereof, vitamin B and esters thereof, vitamin C and esters thereof, vitamin D and esters thereof, vitamin E and esters thereof, for example, vitamin E acetate, zinc, and any combinations thereof. The stabilizing agent may be about 0.2 to 30% (w/v), about 0.5 to 30% (w/v), about 0.5 to 20% (w/v), about 0.5 to 10% (w/v), 1 to 30% (w/v), 1 to 25% (w/v), 1 to 20% (w/v), 1 to 15% (w/v), 2 to 20% (w/v), 2 to 15% (w/v), or 2 to 10% (w/v) of the liquid formulation.

In one embodiment, the stabilizing agent may be a stabilizing agent which substantially does not comprise albumin. Human serum albumin, which is available as a stabilizing agent for proteins, has a possibility of contamination by pathogenic viruses from humans since the human serum albumin is prepared from human blood, and gelatin or bovine serum albumin may cause diseases, or allegic responses in some patients. The albumin-free stabilizing agent according to the present disclosure substantially does not comprise heterogeneous proteins such as a human- or animal-derived serum albumin or purified gelatin, and thus there is no risk of viral infection.

The expression “substantially does not comprise” herein means that the stated material is included to the extent that the material does not contribute to the preparation or activity of the composition, or the characteristics or activity of the formulation, or that the material is not included at all.

While not wishing to be bound by theory, the stabilizing agent may improve the stability of the formulation as described above. Therefore, the remaining rate may vary according to the physical or chemical environment which is altered due to the use of a certain amount of these stabilizing agents.

Surfactant

In one embodiment, the liquid formulation may include a surfactant.

The term “surfactant” herein may generally mean an agent which protects proteins from the strain induced by an air-solution interface and strain induced by a solution-surface interface. For example, the surfactant may protect the protein from aggregation. Examples of polysorbate-based non-ionic surfactants, which are suitable surfactants, may include Polysorbate 20, Polysorbate 40, Polysorbate 60, or Polysorbate 80. Examples of the surfactant may include Poloxamers such as Poloxamer 188, Tweens such as Tween 20 and Tween 80, polyoxyethylene alkyl ethers, Triton X-100, Brij 30, or Brij 35.

When a polysorbate-based non-ionic surfactant is included at a final concentration of 5% or greater (w/v) of the entire solution, this may considerably affect stability of the liquid formulation including eflapegrastim. As a measure of the stability, the remaining rate may be applied. For example, such formulations are understood to have very good stability when the remaing rate of the protein is maintained at about 97% or greater after a 4-week storage test under accelerated conditions (25±2° C./60±5% RH).

Since the first approval in Europe, polysorbate-based non-ionic surfactants have widely been used as an additive in the field of pharmaceuticals/cosmetics, but recently, there have been some reports indicating that such surfactants have a negative effect on the human body. For example, it has been reported that Polysorbate 80 causes anaphylaxis when injected into the human body (Palacios Castano M I et al., Anaphylaxis Due to the Excipient Polysorbate 80, 2016). Therefore, there are a need to control the concentration of a polysorbate-based non-ionic surfactant within a range that does not affect stability of a protein drug or cause patient discomfort, and a need to prevent technically an inevitable increase in concentration of the polysorbate-based non-ionic surfactant during a preparation process of the liquid formulation.

In one or more embodiments, the polysorbate-based non-ionic surfactant may be included at a final concentration of, with respect to a total volume of the entire solution, about 0.0001 to 5% (w/v), about 0.0001 to 0.5% (w/v), about 0.0001 to 0.05% (w/v), about 0.0001 to 0.005% (w/v), about 0.0001 to 0.0005% (w/v), 0.001 to 5% (w/v), 0.001 to 0.5% (w/v), about 0.001 to 0.05% (w/v), about 0.001 to 0.005% (w/v), about 0.01 to 5% (w/v), about 0.01 to 0.5% (w/v), about 0.01 to 0.05% (w/v), about 0.1 to 5% (w/v), or about 0.1 to 0.5% (w/v), and in some other embodiments, about 0.0001 to 4.5% (w/v), 0.0001 to 0.45% (w/v), 0.0001 to 0.045% (w/v), 0.0001 to 0.0045% (w/v), 0.0001 to 0.00045% (w/v), 0.001 to 4.5% (w/v), 0.001 to 0.45% (w/v), 0.001 to 0.045% (w/v), 0.001 to 0.0045% (w/v), 0.01 to 4.5% (w/v), 0.01 to 0.45% (w/v), 0.01 to 0.045% (w/v), 0.1 to 4.5% (w/v), or 0.1 to 0.45% (w/v).

The term “final concentration” herein refers to an actual concentration that is substantially included in the liquid formulation, a concept distinct from a stated concentration. For example, in preparing the liquid formulation, during a process of concentrating eflapegrastim to a target concentration after exchange of a buffer material, the final concentration of the surfactant in the liquid formulation may become higher than the stated concentration. For example, in an embodiment using a polysorbate-based non-ionic surfactant as the surfactant, if the concentration of the surfactant is stated as 0.005% (w/v), the actual concentration or final concentration of the polysorbate-based surfactant in the formulation may be at least 1000 times higher than the stated concentration of 0.005% (w/v), as a result of the polysorbate-based surfactant being concentrated together with eflapegrastim. For example, in KR 10-1340710 (Publication date: Dec. 12, 2013) disclosing a liquid formulation including a granulocyte colony-stimulating factor (G-CSF) conjugate, not eflapegrastim, 0.005% (w/v) or 0.01% (w/v) of Polysorbate 80 is stated in examples, but its final concentration (actual concentation) may exceed at least 5% (w/v) or 10% (w/v). Meanwhile, in a case where the final concentration of the polysorbate-based non-ionic surfactant is stated to be 0.005% (w/v), this means that the formulation is obtained through an exchange of buffer material which substantially does not comprise the polysorbate-based non-ionic surfactant, for example, by dialysis filtration, which is then followed by concentrating the buffer material until a target concentration of eflapegrastim is reached, and then by spiking with the polysorbate-based non-ionic surfactant to 0.005% (w/v). Therefore, the liquid formulation according to one or more embodiments may be pre-treated using a purified column, and the pre-treated liquid formulation may then be concentrated after buffer exchange with a buffer material which substantially does not include the polysorbate-based non-ionic surfactant. As such, though not wishing to be bound by a particular theory, the liquid formulation according to the present disclosure which contains a high concentration of non-natural protein, which is originally highly aggregable, may have significantly improved formulation stability and other improved characteristics, as compared to other liquid formulations.

Tonicity Modifier

In one embodiment, the liquid formulation may include a tonicity modifier.

The term “tonicity modifier” herein may mean a compound or compounds which can be sued to modify the tonicity of the liquid formulation.

The tonicity modifier may include at least one selected from the group consisting of pharmaceutically acceptable salts, sugars, and amino acids. Specifically, the tonicity modifier may be at least one selected from the group consisting of sodium chloride, sodium phosphate, sodium succinate, sodium sulfate, potassium chloride, magnesium chloride, magnesium sulfate, and magnesium chloride, and more specifically, may be sodium chloride. The tonicity modifier may be at least one selected from the group consisting of monosaccharides, disaccharides, oligosaccharides, and polysaccharides, for example, may be at least one selected from the group consisting of trehalose, sucrose, mannitol, sorbitol, fructose, maltose, lactose, and dextran. The tonicity modifier may be at least one selected from the group consisting of proline, alanine, arginine (for example, L-arginine), asparagine, aspartic acid, (for example, L-aspartic acid), glycine, serine, lysine, and histidine.

The concentration of the tonicity modifier at which the formulation according to the present disclosure can be stabilized may be within the range in which the osmolarity can be maintained or controlled. For example, the concentration of the tonicity modifier may be, for example, about 1 to 600 mM, about 5 to 600 mM, about 5 to 400 mM, about 5 to 300 mM, about 10 to 400 mM, about 10 to 300 mM, about 10 to 200 mM, about 20 to 400 mM, about 20 to 200 mM, about 30 to 400 mM, about 30 to 200 mM, about 50 to 600 mM, about 50 to 400 mM, about 80 to 400 mM, about 80 to 200 mM, about 100 to 400 mM, about 100 to 300 mM, or about 100 to 200 mM.

The tonicity modifier may be added in an amount sufficient to provide a suitable osmolarity which will be described later.

Buffer Material and pH

The term “buffer material” herein may mean at least one ingredient capable of protecting a solution from a pH change when an acid or alkali is added to the aqueous solution or when the solution is diluted with a solvent solution. The buffer material may be any material capable of adjusting the pH of the formulation to stabilize the formulation, for example, may be an organic acid buffer or an inorganic acid buffer, for example, an organic acid, an inorganic acid, or a salt of an organic acid or inorganic acid. More specifically, the buffer may be an organic acid or inorganic acid of at least one selected from the group consisting of succinic acid, acetic acid, citric acid, histidine, phosphoric acid, glycine, lactic acid, Tris, or Bis-tris, or may be at least one selected from sodium salt, succinate, acetate, citrate, phosphate, and lactate of the above-listed organic acids or inorganic acids. More specifically, examples of the buffer material may include any pharmaceutically acceptable pH buffer materials known in the art, including alkali salts (sodium or potassium phosphate or hydrogen or di-hydrogen salts thereof), sodium citrate/citric acid, sodium acetate/acetic acid, and mixtures thereof.

In one embodiment, the concentration of the buffer material may be about 5 to 100 mM, about 5 to 80 mM, about 10 to 80 mM, about 10 to 60 mM, about 10 to 50 mM, or about 15 to 25 mM.

In one embodiment, the liquid formulation may be in a pH range of about pH 4 to 8, about pH 5 to 8, about pH 5 to 7, or about pH 5 to 6. For example, the liquid formulation may have a pH of 5.5.

The buffer material may be dissolved in a liquid medium such as water and used in a liquid state having a pH of 4 to 8, a pH of 5 to 8, a pH of 5 to 7, or a pH of 5 to 6.

Osmolarity

According to various documents, the osmolarity of drugs can be controlled to be about 300±30 mOsm/kg. However, since various excipients need to be used, drugs may be also prepared as a hypertonic solution in the technology industry. For intravenous or endovascular administration, generally the upper limit of osmolarity has been suggested not to exceed 1000 mOsm/kg, and the lower limit of osmolarity to exceed 100 mOsm/L or 200 mOsm/L, because the osmolarity of serum is about 285 mOsm/L. Accordingly, in general it is expected that patients are able to withstand an osmolarity of about 100 to 1000 mOsm/kg, about 200 to 1000 mOsm/kg, about 100 to 800 mOsm/kg, or about 200 to 800 mOsm/kg. In one embodiment, the osmolarity of the liquid formulation to exhibit an excellent stabilizing effect may be 400 to 800 mOsm/kg.

The osmolarity can be measured using any measurement method and measurement apparatus known in the art.

While not wishing to be bound by theory, the osmolarity of the liquid formulation according to the present disclosure is thought to be affected by the concentration of eflapegrastim or the concentration of a stabilizing agent or a tonicity modifier, which may be additionally included. As the stability of the liquid formulation according to the present disclosure is increased due to the eflapegrastim in a certain concentration range and/or the hG-CSF variant having a specific amino acid sequence, the composition of the liquid formulation, which has an influence on stability, may be more flexibly adjusted, as compared with the composition of the existing liquid formulation. Accordingly, it may be possible to more flexibly control the composition of the aqueous formulation to have an osmolarity at which effects of the present disclosure can be achieved. For example, the osmolarity of the liquid formulation may be controlled by relatively reducing the concentration of the stabilizing agent or tonicity modifier, which can be additionally included for stability of the liquid formulation.

Conductivity

In another embodiment, the liquid formulation according to the present disclosure may have a conductivity of about 20 mS/cm or less, about 19 mS/cm or less, about 18 mS/cm or less, about 17 mS/cm or less, about 16 mS/cm or less, or about 15 mS/cm or less. Ranges including these mentioned conductivity values, for example, a range from 1 to 20 mS/cm, is also within the scope of the present disclosure. For example, numerical ranges with a combination of the mentioned conductivity values as the upper limit and/or lower limit are within the scope of the present disclosure. Any numerical values within the mentioned numerical ranges, for example, a conductivity value of 1 mS/cm, 2 mS/cm, 3 mS/cm, 4 mS/cm, 5 mS/cm, 6 mS/cm, 7 mS/cm, 8 mS/cm, 9 mS/cm, 10 mS/cm, 11 mS/cm, 12 mS/cm, 13 mS/cm, 14 mS/cm, 15 mS/cm, 16 mS/cm, 17 mS/cm, 18 mS/cm, 19 mS/cm, or 20 mS/cm is within the scope of the present disclosure.

The term “conductivity” herein refers to the ability of the aqueous solution to conduct an electric current between two electrodes. Generally, electrical conductivity and specific conductivity are measures of the ability of a material to conduct an electric current. In a solution, the current flows by ionic transport. Consequently, as the amount of ions present in the aqueous solution increases, the solution may have a higher conductivity. The unit of measure for conductivity is mmhos (mS/cm), and can be measured using a conductivity meter which is comically purchasable.

Maximum Gliding Force and Viscosity

The term “maximum gliding force (MGF)” herein refers to the maximum force exerted when a drug is injected using a syringe, and is influenced by the viscosity of the formulation, injection velocity, and syringe characteristics. The rheological properties of a protein formulation are influenced by the concentration of the protein. In addition, thin needles of 29 G or greater substantially increase maximum gliding force. Eventually, aggregation due to high protein concentration, and consequential viscosity increase may lead to an increase in maximum gliding force, thus raising the need for injection with a needle of less than 29 G to reduce the maximum gliding force, but which may cause discomfort to patients. Reportedly, in general, patients can withstand a maximum gliding force of about 15 N to 20 N (Development of Syringeability Guide for Subcutaneous Protein Formulations, L. Joseph et al., Pfizer Global Research & Development, 2010).

In one embodiment, to provide a therapeutic effect, the liquid formulation, as well containing a sufficient concentration of eflapegrastim, may have a maximum gliding force of about 7N or less, about 6N or less, about 5N or less, about 4N or less, or about 3N or less, as administered with a 29-gauge syringe at a velocity of about 2.835 mm/s. The liquid formulation may have a maximum gliding force of about 10 N or less, about 9N or less, about 8N or less, about 7N or less, about 6N or less, about 5N or less, about 4N or less, about 3.5 N or less, or about 3N or less, as administered with a 29-gauge syringe at a velocity of about 4.725 mm/s. The maximum gliding force may be measured using any gliding force measuring device known in the art, for example, a rheometer commercially available from DAEGO TRADING CO. (Seoul, South Korea). In one embodiment, the liquid formulation according to the present disclosure may have a viscosity of about 10 cP or less, about 9 cP or less, about 8 cP or less, about 7 cP or less, about 6 cP or less, about 5 cP or less, about 4 cP or less, about 3 cP or less, about 3.5 cP or less, about 3 cP or less, about 2.5 cP or less, or about 2 cP or less at an ambient temperature of about 20° C. to 25° C. The viscosity may be measured using any measurement method and device known in the art.

As described above, the maximum gliding force is influenced by the viscosity of a formulation, and the viscosity, i.e., rheological properties of the protein formulation, is influenced by the concentration. Accordingly, the maximum gliding force of the liquid formulation may be influenced by the remaining rate and concentration of the protein drug (eflapegrastim), or a buffer material, and may also be influenced by a surfactant, a stabilizing agent, or a tonicity modifier which can be additionally included.

Patient Friendly Formulation

For a therapeutic effect, a protein drug is required to be formulated at a high concentration. However, with an increasing protein concentration, problems such as aggregation, insolubility and degradation also increasingly occur. Therefore, in a pharmaceutical protein formulation, balancing the ingredients and concentrations thereof to improve stability and therapeutic requirements is one of the technical issues to be addressed in the art. Accordingly, in the present disclosure, it was possible to prepare a formulation having high stability and solubility as well as containing a high concentration of active ingredient. In addition to these surprising advances of the protein formulation according to the present disclosure for therapeutic use, irritation/pain and discomfort at the site of administration when such a liquid formulation of protein drug is administered are still challenges to be addressed.

Osmolarity and Irritation/Pain at Administration Site

Osmolarity changes in tissues and cells are perceived as serious signals in the human body, activating dendritic cells and stimulating immune and inflammatory responses (Gallo and Gallucci, 2013). It has been reported that hypertonicity in the digestive tract of human infants may cause necrotizing colitis (Atakent et al., 1984). It is well known that the presence of pain receptors is the cause of feeling of pains induced by various events including injections. Peripheral pain sensations are mediated through afferent fibers (sensory nerve fibers) called nociceptors (Brazeau at al., 1998). Functionally, nociceptors are classified into two main types, polymodal nociceptors that respond to chemicals, and mechanothermal nociceptors that respond to mechanical and thermal stimuli. Thus, the sensitivity of nociceptors to pain is dependent not only on the types of chemicals, but also on the injection location, injection velocity, and injection volume. Hypertonic solutions (or hypotonic solutions) can draw water out of cells (or cause water absorption into cells), and activate compression (or stretch)-sensitive channels, thus causing pain.

Maximum Gliding Force. Viscosity, and Patient's Uncomfortableness

With respect to reducing the injection volume (injection volume of a drug during injection) and securing storage space, higher-concentration protein formulations are drawing attention. There are several practical problems with the development of high-concentration protein formulations, in terms of stability, preparation, and delivery, due to the tendency of proteins to aggregate at high concentrations. The physical properties of high-concentration protein formulations affect the transportability of the proteins, and such a high-concentration solution often has high viscosity, which may prevent the solution from passing through a syringe needle. Therefore, the viscosity of a material and the protein concentration are correlated, and formulations having higher protein concentrations are also higher in viscosity, and thus cause discomfort to patients. Therefore, the development of a formulation having improved patient acceptance, as well as having increased therapeutic effects due to an increase in the concentration of a protein formulation and reduction in viscosity may present an advance in designing protein formulations.

To reduce the maximum gliding force and increase therapeutic effects of a protein formulation, and further to administer the protein formulation with a thinner needle, the protein formulation needs to be less aggregated while containing a high concentration of protein, and to have low viscosity. These conflicting properties (i.e., low viscosity and high protein concentration) must be resolved.

Patient Friendly (PF) Index

As described above, the production of high-concentration protein formulations may give rise to significant problems with respect to opalescence, aggregation, and precipitation. In addition to the possibility of non-natural protein aggregation and microparticle formation, reversible autobinding may also occur, leading to viscosity increases and other properties that complicate delivery by injection. High viscosity may also complicate the preparation of high-protein formulations by means of filtration. Therefore, in the development of high-stability, patient-friendly formulations, various factors need to be considered carefully in combination. That is, the remaining rate, which is a factor related with the stability of formulations, is influenced by a stabilizing agent and a surfactant, and consequentially affects the viscosity of the formulation, and then the maximum gliding force. In addition, the osmolarity is influenced by a tonicity modifier and a buffer material, and also affects conductivity.

In one embodiment, the liquid formulation may satisfy a specific range of a patient-friendly (PF) index represented by Equation 1:

Patient-friendly (PF) index=Osm (mOsm/kg)/100+MGF (N) [Equation 1]

In Equation 1, Osm indicates the osmolarity value of the liquid formulation, and MGF indicates a value of maximum gliding force (MGF) when the liquid formulation is injected with a 29-gauge (29 G) syringe at a velocity of 2.835 mm/s.

The PF index may be 10 or smaller, provided that the osmolarity and the maximum gliding force are appropriate. When the PF index exceeds 10, due to a difference in osmolarity from that of the body fluid and/or a high maximum gliding force, patient discomfort may increase rapidly. The PF index may be 3 or greater, provided that the osmolarity and the maximum gliding force are appropriate. When the PF index is less than 3, due to a difference in osmolarity from that of the body fluid and/or a low maximum gliding force, patient discomfort may increase rapidly.

In particular, the PF index of the liquid formulation may be in a range of from 3 to 10, from 5 to 10, or from 6 to 10, for example, may be from 6 to 9. Considering that in most cases the maximum gliding force affected by viscosity is at least 1N or greater, when the osmolarity is 1000 mOsm/kg, the PF index may exceed 10, and thus it may be difficult to achieve patient-friendly injection. In addition, considering that in most cases the maximum gliding force is 1N or grater, when the osmolarity is less than 200 mOsm/kg, the PF index may not exceed 3, and thus it may be difficult to achieve patient-friendly injection.

Therefore, since a liquid formulation with a PF index within the above-described ranges may have a low viscosity, a low gliding force, and an appropriate osmolarity in consideration of the formulation being a high-concentration protein formulation, it is possible to resolve the above-described technical problems, thereby allowing high formulation stability and patient-friendly injection to be achieved.

In one or more embodiments, the liquid formulation may include about 11 to 66 mg/mL of eflapegrastim and about 5 to 100 mM of the buffer material, or may include about 11 to 66 mg/mL of eflapegrastim, about 5 to 100 mM of the buffer material, and about 0.001 to 5% (w/v) of a polysorbate-based non-ionic surfactant. In one embodiment, the liquid formulation may include about 11 to 66 mg/mL of eflapegrastim, about 5 to 100 mM of a buffer material, about 1 to 20% (w/v) of a stabilizing agent, about 0.001 to 0.5% (w/v) of a surfactant, and about 5 to 200 mM of a tonicity modifier. For example, the liquid formulation may include about 11 to 66 mg/mL of eflapegrastim and about 5 to 100 mM of sodium citrate, or may include about 11 to 66 mg/mL of eflapegrastim, about 5 to 100 mM of sodium citrate, and about 0.001 to 5% (w/v) of Polysorbate 80. In one embodiment, the liquid formulation may include about 11 to 66 mg/mL of eflapegrastim, about 5 to 100 mM of sodium citrate, about 1 to 20% (w/v) of mannitol, about 0.001 to 5% (w/v) of Polysorbate 80, and about 5 to 200 mM of sodium chloride, or may include about 11 to 66 mg/mL of eflapegrastim, about 5 to 100 mM of sodium citrate, about 1 to 20% (w/v) of mannitol, about 0.001 to 0.5% (w/v) of Polysorbate 80, and 5 to 200 mM of sodium chloride.

The injection volume of the liquid formulation according to the embodiments may be appropriately controlled in consideration of a reduction in irritation/pain at an administration site or patient discomfort. For example, the liquid formulation may have an injection volume of about 0.2 to 1.2 mL.

In another aspect of the present disclosure, there is provided an article of manufacture including the liquid formulation according to any of the embodiments.

In another embodiment of the present disclosure, an article of manufacture may include a drug formulation and instructions for use thereof. The prepared product may include a container. Suitable examples of the container may be a bottle, a vial, and a test tube. The container may be prepared from various materials, for example, glass, plastic, or metal.

One or more embodiments of the present disclosure will now be described in detail with reference to the following examples. However, these examples are only for illustrative purposes and are not intended to limit the scope of the one or more embodiments of the present disclosure.

### Example 1. Analysis of Patient-Friendly Injectable Liquid Formulation

This example was to derive a patient-friendly injectable formulation as a final liquid formulation by applying a variety of parameters of the prepared formulations that affect patients when the formulations were administered. To this end, as described above, an appropriate level of osmolarity for the liquid formulation according to embodiments was determined to be 100 to 1000 mOsm/kg, for example, 200 to 1000 mOsm/kg, based on the disclosure in “Tolerability of hypertonic injectables, Wei Wang, International Journal of Pharmaceutics 490 (2015) 308-315”, and “Tonicity Agents Clarity—American Pharmacists Association.” An appropriate level of the maximum gliding force was calculated as 5N or less, with reference to the disclosure in “Development of Syringeability Guide for Subcutaneous Protein Formulations, L. Joseph et al., Pfizer Global Research & Development, 2010.”

In consideration of the complementarity between these values, a parameter was introduced and represented by Equation 1. It was found that when the conditions of Equation 1 are satisfied, a liquid formulation having high patient-friendliness may be obtained, and a resulting value from Equation 1 was named as the patient-friendly (PF) index.

Patient-friendly (PF) index=Osm(mOsm/kg)/100+MGF(N) [Equation 1]

In Equation 1, Osm indicates the osmolarity of the liquid formulation, and MGF indicates the maximum gliding force when the liquid formulation is administered with a 29-gauge (29 G) syringe at a velocity of 2.835 mm/s.

The PF index was determined to be 3 to 10, provided that the osmolarity and the maximum gliding force are appropriate.

Considering that in most cases the maximum gliding force is at least 1N or greater, when the osmolarity of the liquid formulation is greater than 1000 mOsm/kg, the PF index may exceed 10, and thus it may be difficult to achieve patient-friendly injection.

In addition, considering that mostly the maximum gliding force is 1N or greater, when the osmolarity is less than 200 mOsm/kg, the PF index may not exceed 3, and thus it may be difficult to achieve patient-friendly injection.

That is, when the PF index is 3 to 10, the liquid formulation is considered to be an excellent patient-friendly injectable formulation.

Therefore, since a liquid formulation with a PF index within the ranges described above may have a low viscosity, a low gliding force, and an appropriate osmolarity in consideration of the formulation being a high-concentration protein formulation, it is possible to solve the technical problems described above, thereby allowing high formulation stability and patient-friendly injection to be achieved.

### Example 2. Analysis of Stability of Liquid Formulation

The stability of the high-concentration protein formulation was measured from the remaining rate after a 4-week storage test.

In particular, to measure the remaining rate of the liquid formulation, the remaining rate of eflapegrastim after a 4-week storage test under accelerated conditions (25±2° C./60±5% RH) was measured using reversed phase high-performance liquid chromatography (RP-HPLC) and size-exclusion high-performance liquid chromatography (SE-HPLC).

An n^{th}-week remaining rate of eflapegrastim in the liquid formulation was calculated by Equation 2.

n^{th}-week remaining rate (%)=Purity value at n^{th} week/Initial purity value×100 [Equation 2]

The purity is a relative ratio of main peaks according to HPLC.

The HPLCs used were Agilent 1200 series. RP-HPLC was carried out using a Phenomenex Jupiter C4 column at 60° C. A two-eluent linear gradient system was used at a flow rate of 1.0 mL/min, using mobile phase A containing 0.1% trifluoroacetic acid in 20% acetonitrile, and mobile phase B containing 0.1% trifluoroacetic acid in 80% acetonitrile. After initial stabilization for at least one hour with 76% of mobile phase A and 24% of mobile phase B, the linear gradient system was operated with 24% to 60% of mobile phase B for 0 to 15 minutes, 60% to 73% of mobile phase B for 15 to 48 minutes, and 73% to 100% of mobile phase B for 48 to 75 minutes, and then re-equilibrated with 24% of mobile phase B for 75 to 85 minutes. The injection volume of samples was 20 ug, the detector was set at a wavelength of 214 nm, and all the procedures were controlled with Agilent Chemstation software.

SE-HPLC was carried out using a Shodex Protein KW-803 column in ambient temperature conditions. An isocratic gradient system was used at a flow rate of 0.6 mL/min, using a mobile phase containing 50 mM of sodium phosphate, 150 mM of sodium chloride, and 5% of isopropyl alcohol. After stabilization for at least one hour, the analysis was carried out for 60 minutes. The injection volume of samples was 20 ug, the detector was set at a wavelength of 214 nm, and all the procedures were controlled with Agilent Chemstation software.

In consideration of the physicochemical characteristics of eflapegrastim and common knowledge in the field of protein formulations, a liquid formulation was found to have stability as follows, when the remaining rate of eflapegrastim was maintained after a 4-week storage test in accelerated conditions (25±2° C./60±5% RH):

95% or greater maintained: good stability

97% or greater maintained; very good stability

98% or greater maintained: excellent stability

### Preparation Examples 1 to 46. Preparation of Eflapegrastim-Containing Liquid Formulations

Liquid formulations containing high-concentration eflapegrastim, being patient-friendly, and having formulation stability, as presented in Examples 1 and 2, were designed.

Liquid formulations of Preparation Examples 1 to 36, which were considered to likely ensure formulation stability and patient-friendly injection through prediction by simulation prediction, were prepared. In addition, to check formulation stability when a high-concentration polysorbate-based non-ionic surfactant was included according to a preparation method of the related art, liquid formulations of Preparation Examples 37 to 39 were prepared. In addition, liquid formulations of Preparation Examples 40 to 43, expected to cause patient discomfort or to have a formulation stability problem, were prepared. To check, as a major factor that affects formulation stability, changes according to concentration of a polysorbate-based non-ionic surfactant, liquid formulations of Preparation Examples 44 to 46 were prepared.

### 1. Preparation Examples 1 to 36

Liquid formulations including eflapegrastim were prepared as follows.

First, a liquid formulation including 22 mg/mL of eflapegrastim, 20 mM of sodium citrate (pH 5.5), 5% of mannitol, 150 mM of sodium chloride, and 0.005% (w/v, final concentration) of Polysorbate 80 was prepared. Then, for sample pre-treatment, Polysorbate 80 was removed from the prepared liquid formulation using a S.Q purification column (Source 15Q, GE Healthcare). Then, only the most major fractions were recovered from the resulting purification profile. Then, the pre-treated liquid formulation was subjected to buffer exchange by filtration. In particular, buffer exchanging was performed using VivaSpin 20 (Sartorius) in a buffer containing no Polysorbate 80, at 3,700 rpm, five times in total for 1 hour. Then, the buffer-exchanged liquid formulation was concentrated to be 2 times or 3 times a target concentration. In consideration of a final volume and a target concentration, Polysorbate 80-free buffer was added to the concentrated liquid formulation. The concentrated liquid formulation was then exposed to spiking using a Polysorbate 80 stock concentrated 100 times higher than the target concentration, thereby preparing a final liquid formulation having a final polysorbate concentration of 0.005% (w/v). In addition, in Preparation Examples 2 to 37, liquid formulations were prepared in the same manner as in Example 1 but in different compositions from that of the formulation of Preparation Example 1.

That is, the compositions of the liquid formulations of Preparation Examples 1 to 36 were as follows.

### Preparation Example 1

22 mg/mL of eflapegrastim, 20 mM of sodium citrate (pH 5.5), 5% (w/v) of mannitol, 150 mM of sodium chloride, and 0.005% (w/v, final concentration) of Polysorbate 80.

### Preparation Example 2

11 mg/mL of eflapegrastim, 20 mM of sodium citrate (pH 5.5), 5% (w/v) of mannitol, 150 mM of sodium chloride, and 0.005% (w/v, final concentration) of Polysorbate 80.

### Preparation Example 3

44 mg/mL of eflapegrastim, 20 mM of sodium citrate (pH 5.5), 5% (w/v) of mannitol, 150 mM of sodium chloride, and 0.005% (w/v, final concentration) of Polysorbate 80.

### Preparation Example 4

66 mg/mL of eflapegrastim, 20 mM of sodium citrate (pH 5.5), 5% (w/v) of mannitol, 150 mM of sodium chloride, and 0.005% (w/v, final concentration) of Polysorbate 80.

### Preparation Example 5

22 mg/mL of eflapegrastim, 20 mM of sodium citrate (pH 5.5), 1% (w/v) of mannitol, 10 mM of sodium chloride, and 0.005% (w/v, final concentration) of Polysorbate 80.

### Preparation Example 6

22 mg/mL of eflapegrastim, 20 mM of sodium citrate (pH 5.5), 3% (w/v) of mannitol, 50 mM of sodium chloride, and 0.005% (w/v, final concentration) of Polysorbate 80.

### Preparation Example 7

22 mg/mL of eflapegrastim, 20 mM of sodium acetate (pH 5.5), 5% (w/v) of sorbitol, 150 mM of sodium chloride, and 0.005% (w/v, final concentration) of Polysorbate 80.

### Preparation Example 8

44 mg/mL of eflapegrastim, 20 mM of sodium acetate (pH 5.5), 5% (w/v) of sorbitol, 150 mM of sodium chloride, and 0.005% (w/v, final concentration) of Polysorbate 80.

### Preparation Example 9

66 mg/mL of eflapegrastim, 20 mM of sodium acetate (pH 5.5), 5% (w/v) of sorbitol, 150 mM of sodium chloride, and 0.005% (w/v, final concentration) of Polysorbate 80.

### Preparation Example 10

22 mg/mL of eflapegrastim, 20 mM of sodium acetate (pH 5.5), 5% (w/v) of sucrose, 150 mM of sodium chloride, and 0.005% (w/v, final concentration) of Polysorbate 80.

### Preparation Example 11

44 mg/mL of eflapegrastim, 20 mM of sodium acetate (pH 5.5), 5% (w/v) of sucrose, 150 mM of sodium chloride, and 0.005% (w/v, final concentration) of Polysorbate 80.

### Preparation Example 12

66 mg/mL of eflapegrastim, 20 mM of sodium acetate (pH 5.5), 5% (w/v) of sucrose, 150 mM of sodium chloride, and 0.005% (w/v, final concentration) of Polysorbate 80.

### Preparation Example 13

22 mg/mL of eflapegrastim, 20 mM of sodium acetate (pH 5.5), 3% (w/v) of proline, 150 mM of sodium chloride, and 0.005% (w/v, final concentration) of Polysorbate 80.

### Preparation Example 14

44 mg/mL of eflapegrastim, 20 mM of sodium acetate (pH 5.5), 3% (w/v) of proline, 150 mM of sodium chloride, and 0.005% (w/v, final concentration) of Polysorbate 80.

### Preparation Example 15

66 mg/mL of eflapegrastim, 20 mM of sodium acetate (pH 5.5), 3% (w/v) of proline, 150 mM of sodium chloride, and 0.005% (w/v, final concentration) of Polysorbate 80.

### Preparation Example 16

22 mg/mL of eflapegrastim, 20 mM of histidine (pH 5.5), 5% (w/v) of sorbitol, 150 mM of sodium chloride, and 0.005% (w/v, final concentration) of Polysorbate 80.

### Preparation Example 17

44 mg/mL of eflapegrastim, 20 mM of histidine (pH 5.5), 5% (w/v) of sorbitol, 150 mM of sodium chloride, and 0.005% (w/v, final concentration) of Polysorbate 80.

### Preparation Example 18

66 mg/mL of eflapegrastim, 20 mM of histidine (pH 5.5), 5% (w/v) of sorbitol, 150 mM of sodium chloride, and 0.005% (w/v, final concentration) of Polysorbate 80.

### Preparation Example 19

22 mg/mL of eflapegrastim, 20 mM of histidine (pH 5.5), 5% (w/v) of sucrose, 150 mM of sodium chloride, and 0.005% (w/v, final concentration) of Polysorbate 80.

### Preparation Example 20

44 mg/mL of eflapegrastim, 20 mM of histidine (pH 5.5), 5% (w/v) of sucrose, 150 mM of sodium chloride, and 0.005% (w/v, final concentration) of Polysorbate 80.

### Preparation Example 21

66 mg/mL of eflapegrastim, 20 mM of histidine (pH 5.5), 5% (w/v) of sucrose, 150 mM of sodium chloride, and 0.005% (w/v, final concentration) of Polysorbate 80.

### Preparation Example 22

22 mg/mL of eflapegrastim, 20 mM of histidine (pH 5.5), 3% (w/v) of proline, 150 mM of sodium chloride, and 0.005% (w/v, final concentration) of Polysorbate 80.

### Preparation Example 23

44 mg/mL of eflapegrastim, 20 mM of histidine (pH 5.5), 3% (w/v) of proline, 150 mM of sodium chloride, and 0.005% (w/v, final concentration) of Polysorbate 80.

### Preparation Example 24

66 mg/mL of eflapegrastim, 20 mM of histidine (pH 5.5), 3% (w/v) of proline, 150 mM of sodium chloride, and 0.005% (w/v, final concentration) of Polysorbate 80.

### Preparation Example 25

22 mg/mL of eflapegrastim, 20 mM of sodium citrate (pH 5.5), 5% (w/v) of mannitol, 20 mM of sodium phosphate, and 0.01% (w/v, final concentration) of Polysorbate 80.

### Preparation Example 26

44 mg/mL of eflapegrastim, 20 mM of sodium citrate (pH 5.5), 5% (w/v) of mannitol, 20 mM of sodium phosphate, and 0.01% (w/v, final concentration) of Polysorbate 80.

### Preparation Example 27

66 mg/mL of eflapegrastim, 20 mM of sodium citrate (pH 5.5), 5% (w/v) of mannitol, 20 mM of sodium phosphate, and 0.01% (w/v, final concentration) of Polysorbate 80.

### Preparation Example 28

22 mg/mL of eflapegrastim, 20 mM of sodium citrate (pH 5.5), 5% (w/v) of mannitol, 25 mM of arginine, 20 mM of histidine, and 0.2% (w/v, final concentration) of Polysorbate 80.

### Preparation Example 29

44 mg/mL of eflapegrastim, 20 mM of sodium citrate (pH 5.5), 5% (w/v) of mannitol, 25 mM of arginine, 20 mM of histidine, and 0.2% (w/v, final concentration) of Polysorbate 80.

### Preparation Example 30

66 mg/mL of eflapegrastim, 20 mM of sodium citrate (pH 5.5), 5% (w/v) of mannitol, 25 mM of arginine, 20 mM of histidine, and 0.2% (w/v, final concentration) of Polysorbate 80.

### Preparation Example 31

22 mg/mL of eflapegrastim, 20 mM of sodium citrate (pH 5.5), 3% (w/v) of proline, 150 mM of sodium chloride, and 0.01% (w/v, final concentration) of Polysorbate 20.

### Preparation Example 32

44 mg/mL of eflapegrastim, 20 mM of sodium citrate (pH 5.5), 3% (w/v) of proline, 150 mM of sodium chloride, and 0.01% (w/v, final concentration) of Polysorbate 20.

### Preparation Example 33

66 mg/mL of eflapegrastim, 20 mM of sodium citrate (pH 5.5), 3% (w/v) of proline, 150 mM of sodium chloride, and 0.01% (w/v, final concentration) of Polysorbate 20.

### Preparation Example 34

22 mg/mL of eflapegrastim, 20 mM of sodium citrate (pH 5.5), and 125 mM of sodium chloride.

### Preparation Example 35

44 mg/mL of eflapegrastim, 20 mM of sodium citrate (pH 5.5), and 125 mM of sodium chloride.

### Preparation Example 36

66 mg/mL of eflapegrastim, 20 mM of sodium citrate (pH 5.5), and 125 mM of sodium chloride.

### 2. Preparation Examples 37 to 39

A liquid formulation of Preparation Example 37 including eflapegrastim was prepared with reference to Korean Patent No. 10-1340710, wherein, as an active ingredient, eflapegrastim including human granulocyte-colony stimulating factor (hG-CSF) variant having a different amino acid sequence from that disclosed in Korean Patent No. 10-1340710, was used and the concentration thereof was also varied.

In particular, a liquid formulation was prepared using 22 mg/mL of eflapegrastim, 20 mM of sodium citrate (pH 5.5), 5% of mannitol, 150 mM of sodium chloride, and 0.005% (w/v) of Polysorbate 80 (before a concentration process). Then, without pre-treatment of sample, the liquid formulation was immediately subjected to buffer exchange by means of filtration. In particular, buffer exchanging was performed using VivaSpin 20 (Sartorius) in a buffer containing Polysorbate 80, at 3,700 rpm, five times in total for 1 hour. Then, the buffer-exchanged liquid formulation was concentrated to be 2 times of a target concentration. In consideration of a final volume and a target concentration, the concentrated liquid formulation was diluted with a buffer containing all of the excipients, thereby preparing a final liquid formulation. In the liquid formulation of Preparation Example 37, the above-mentioned concentration of Polysorbate 80 was a concentration before a concentration process, but in the following test examples, polysorbate 80 exceeding at least 5% (w/v, final concentration) was applied.

That is, the composition of Preparation Example 37 was as follows.

### Preparation Example 37

22 mg/mL of eflapegrastim, 20 mM of sodium citrate (pH 5.5), 5% (w/v) of mannitol, 150 mM of sodium chloride, and exceeding at least 5% (w/v, final concentration) of Polysorbate 80.

In addition, in Preparation Examples 38 and 39, liquid formulations were prepared in the same manner as in Example 37 but in different compositions from that of the formulation of Preparation Example 37.

### Preparation Example 38

31.5 mg/mL of eflapegrastim, 20 mM of sodium citrate (pH 5.5), 5% (w/v) of mannitol, 150 mM of sodium chloride, and exceeding at least 5% (w/v, final concentration) of Polysorbate 80.

### Preparation Example 39

40 mg/mL of eflapegrastim, 20 mM of sodium citrate (pH 5.5), 5% (w/v) of mannitol, 150 mM of sodium chloride, and exceeding at least 5% (w/v, final concentration) of Polysorbate 80.

### 3. Preparation Examples 40 to 43

Liquid formulations were prepared in the same manner as in Preparation Example 1 by in different compositions as that of the liquid formulation of Preparation Example 1.

That is, the compositions of the liquid formulations of Preparation Examples 40 to 43 were as follows.

### Preparation Example 40

22 mg/mL of eflapegrastim, 20 mM of sodium citrate (pH 5.5), and 0.005% (w/v, final concentration) of Polysorbate 80.

### Preparation Example 41

22 mg/mL of eflapegrastim, 20 mM of sodium citrate (pH 5.5), 10% (w/v) of mannitol, 500 mM of sodium chloride, and 0.005% (w/v, final concentration) of Polysorbate 80.

### Preparation Example 42

22 mg/mL of eflapegrastim, 20 mM of sodium citrate (pH 5.5), 20% (w/v) of glucose, 150 mM of sodium chloride, and 0.005% (w/v, final concentration) of Polysorbate 80.

### Preparation Example 43

22 mg/mL of eflapegrastim, 20 mM of sodium citrate (pH 5.5), 5% (w/v) of gelatin, 150 mM of sodium chloride, and 0.005% (w/v, final concentration) of Polysorbate 80.

### 4. Preparation Examples 44 to 46

Liquid formulations were prepared in the same manner as in Preparation Example 1 by in different compositions as that of the liquid formulation of Preparation Example 1.

That is, the compositions of the liquid formulations of Preparation Examples 44 to 46 were as follows.

### Preparation Example 44

22 mg/mL of eflapegrastim, 20 mM of sodium citrate (pH 5.5), 5% (w/v) of mannitol, 150 mM of sodium chloride, and 0.5% (w/v, final concentration) of Polysorbate 80.

### Preparation Example 45

22 mg/mL of eflapegrastim, 20 mM of sodium citrate (pH 5.5), 5% (w/v) of mannitol, 150 mM of sodium chloride, and 2.5% (w/v, final concentration) of Polysorbate 80.

### Preparation Example 46

22 mg/mL of eflapegrastim, 20 mM of sodium citrate (pH 5.5), 5% (w/v) of mannitol, 150 mM of sodium chloride, and 5% (w/v, final concentration) of Polysorbate 80.

### Test Example 1. Evaluation of Remaining Rate and Patient-Friendly Index of Liquid Formulation

Measurements of osmolarity, conductivity, viscosity, and maximum gliding force were performed on the liquid formulations of Preparation Examples 1 to 6 and Preparation Examples 37 to 43, and thereafter, the remaining rates of eflapegrastim after a 4-week storage test in accelerated conditions (25±2° C./60±5% RH) were measured.

### 1. Test of Liquid Formulation of Preparation Example 1

The liquid formulation of Preparation Example 1 had an osmolarity of 645 mOsm/kg, as measured using an automatic osmometer (Gonotec, OSMOMAT auto).

The conductivity of the liquid formulation of Preparation Example 1 was measured at ambient temperature using a conductivity meter (Compact Conductivity Meter EC33, LAQUAtwin, Horiba) according to instructions of the manufacturer of the system. As a result, the conductivity of the liquid formulation of Preparation Example 1 was 14.37 mS/cm.

The liquid formulation of Preparation Examplel had a viscosity value of 1.86 cP, as measured at room temperature (20 to 25° C.) using a Vibration viscometer (A&D, SV-1A).

The maximum gliding force of the liquid formulation of Preparation Example 1 was measured using a rheometer (Sun scientific, Compac-100) by injecting a liquid formulation with a 29-G syringe (22.68 mm with respect to 400 uL) at a velocity of 4.725 mm/s (500 uL/6 sec) and a velocity of 2.835 mm/s (500 uL/10 sec), and the values were confirmed using a Reology Data System Ver 3.0. The resulting values were 3.099 N and 2.099 N, respectively.

To measure the remaining rate of the liquid formulation of Preparation Example 1, the remaining rate of eflapegrastim after a 4-week storage test under accelerated conditions (25±2° C./60±5% RH) was measured using reversed phase high-performance liquid chromatography (RP-HPLC) and size-exclusion high-performance liquid chromatography (SE-HPLC).

The remaining rates of eflapegrastim measured by RP-HPLC and SE-HPLC are shown in Table 1.

TABLE 1

| | RP-HPLC (Remaining rate, %) | | | | | SE-HPLC (Remaining rate, %) | | | | |
|---|---|---|---|---|---|---|---|---|---|---|
| | 0 W | 1 W | 2 W | 3 W | 4 W | 0 W | 1 W | 2 W | 3 W | 4 W |
| Preparation | 100 | 99.9 | 99.8 | 99.4 | 99.2 | 100 | 100 | 99.7 | 99.6 | 99.5 |
| Example 1 | | | | | | | | | | |

### 2. Test of Liquid Formulation of Preparation Example 2

The osmolarity, conductivity, viscosity, maximum gliding force, and remaining rate of the formulation were measured in the same manner as in Test Example 1.

As a result, the formulation of Preparation Example 2 had an osmolarity of 643.3 mOsm/kg, a conductivity of about 14.80 mS/cm, and a viscosity value of 1.39 cP at room temperature (20 to 25° C.). The formulation of Preparation Example 2 had a maximum gliding force of 2.648 N (at 4.725 mm/s velocity with a 29-G syringe), and 2.285 N (at 2.835 mm/s velocity with a 29-G syringe).

The remaining rates of the formulation of Preparation Example 2 under the accelerated conditions (25±2° C./60±5% RH) are shown in Table 2.

TABLE 2

| | RP-HPLC (Remaining rate, %) | | | | | SE-HPLC (Remaining rate, %) | | | | |
|---|---|---|---|---|---|---|---|---|---|---|
| | 0 W | 1 W | 2 W | 3 W | 4 W | 0 W | 1 W | 2 W | 3 W | 4 W |
| Preparation | 100 | 99.9 | 99.7 | 99.2 | 99.1 | 100 | 100 | 99.6 | 99.4 | 99.0 |
| Example 2 | | | | | | | | | | |

### 3. Test of Liquid Formulation of Preparation Example 3

The osmolarity, conductivity, viscosity, maximum gliding force, and remaining rate of the formulation were measured in the same manner as in Test Example 1

As a result, the formulation of Preparation Example 3 had an osmolarity of 657.7 mOsm/kg, a conductivity of 13.45 mS/cm, and a viscosity value of 2.32 cP at room temperature (20 to 25° C.). The formulation of Preparation Example 3 had a maximum gliding force of 3.177 N (at 4.725 mm/s velocity with a 29-G syringe), and 2.775 N (at 2.835 mm/s velocity with a 29-G syringe).

The remaining rates of the formulation of Preparation Example 3 under the accelerated conditions (25±2° C./60±5% RH) are shown in Table 3.

TABLE 3

| | RP-HPLC (Remaining rate, %) | | | | | SE-HPLC (Remaining rate, %) | | | | |
|---|---|---|---|---|---|---|---|---|---|---|
| | 0 W | 1 W | 2 W | 3 W | 4 W | 0 W | 1 W | 2 W | 3 W | 4 W |
| Preparation | 100 | 99.8 | 99.5 | 99.0 | 98.9 | 100 | 100 | 99.5 | 99.2 | 98.8 |
| Example 3 | | | | | | | | | | |

### 4. Test of Liquid Formulation of Preparation Example 4

The osmolarity, conductivity, viscosity, maximum gliding force, and remaining rate of the formulation were measured in the same manner as in Test Example 1.

As a result, the formulation of Preparation Example 4 had an osmolarity of 679 mOsm/kg, a conductivity of 12.67 mS/cm, and a viscosity value of 3.54 cP at room temperature (20 to 25° C.). The formulation of Preparation Example 4 had a maximum gliding force of 3.815 N (at 4.725 mm/s velocity with a 29-G syringe), and 2.716 N (at 2.835 mm/s velocity with a 29-G syringe).

The remaining rates of the formulation of Preparation Example 4 under the accelerated conditions (25±2° C./60±5% RH) are shown in Table 4.

TABLE 4

| | RP-HPLC (Remaining rate, %) | | | | | SE-HPLC (Remaining rate, %) | | | | |
|---|---|---|---|---|---|---|---|---|---|---|
| | 0 W | 1 W | 2 W | 3 W | 4 W | 0 W | 1 W | 2 W | 3 W | 4 W |
| Preparation | 100 | 99.8 | 99.5 | 99.1 | 98.9 | 100 | 99.9 | 99.2 | 99.0 | 98.6 |
| Example 4 | | | | | | | | | | |

### 5. Test of Liquid Formulation of Preparation Example 5

The osmolarity, conductivity, viscosity, maximum gliding force, and remaining rate of the formulation were measured in the same manner as in Test Example 1.

As a result, the formulation of Preparation Example 5 had an osmolarity of 135.3 mOsm/kg, a conductivity of 4.27 mS/cm, and a viscosity value of 1.40 cP at room temperature (20 to 25° C.). The formulation of Preparation Example 5 had a maximum gliding force of 2.442 N (at 4.725 mm/s velocity with a 29-G syringe), and 1.657 N (at 2.835 mm/s velocity with a 29-G syringe).

The remaining rates of the formulation of Preparation Example 5 under the accelerated conditions (25±2° C./60±5% RH) are shown in Table 5.

TABLE 5

| | RP-HPLC (Remaining rate, %) | | | | | SE-HPLC (Remaining rate, %) | | | | |
|---|---|---|---|---|---|---|---|---|---|---|
| | 0 W | 1 W | 2 W | 3 W | 4 W | 0 W | 1 W | 2 W | 3 W | 4 W |
| Preparation | 100 | 99.8 | 99.5 | 98.9 | 98.8 | 100 | 99.9 | 99.5 | 99.3 | 99.1 |
| Example 5 | | | | | | | | | | |

### 6. Test of Liquid Formulation of Preparation Example 6

The osmolarity, conductivity, viscosity, maximum gliding force, and remaining rate of the formulation were measured in the same manner as in Test Example 1.

As a result, the formulation of Preparation Example 6 had an osmolarity of 334.7 mOsm/kg, a conductivity of 7.49 mS/cm, and a viscosity value of 1.50 cP at room temperature (20 to 25° C.). The formulation of Preparation Example 6 had a maximum gliding force of 2.746 N (at 4.725 mm/s velocity with a 29-G syringe), and 1.285 N (at 2.835 mm/s velocity with a 29-G syringe).

The remaining rates of the formulation of Preparation Example 6 under the accelerated conditions (25±2° C./60±5% RH) are shown in Table 6.

TABLE 6

| | RP-HPLC (Remaining rate, %) | | | | | SE-HPLC (Remaining rate, %) | | | | |
|---|---|---|---|---|---|---|---|---|---|---|
| | 0 W | 1 W | 2 W | 3 W | 4 W | 0 W | 1 W | 2 W | 3 W | 4 W |
| Preparation | 100 | 99.7 | 99.4 | 99.0 | 98.9 | 100 | 100 | 99.1 | 98.9 | 98.7 |
| Example 6 | | | | | | | | | | |

### 7. Test of Liquid Formulation of Preparation Example 37

The remaining rates of the formulation of Preparation Example 37 measured under the accelerated conditions (25±2° C./60±5% RH) as in Test Example 1 are shown in Table 7.

TABLE 7

| | RP-HPLC (Remaining rate, %) | | | | | SE-HPLC (Remaining rate, %) | | | | |
|---|---|---|---|---|---|---|---|---|---|---|
| | 0 W | 1 W | 2 W | 3 W | 4 W | 0 W | 1 W | 2 W | 3 W | 4 W |
| Preparation | 100 | 100 | 98.5 | 96.9 | 94.3 | 100 | 99.7 | 99.0 | 98.0 | 97.0 |
| Example 37 | | | | | | | | | | |

### 8. Test of Liquid Formulation of Preparation Example 38

The remaining rates of the formulation of Preparation Example 38 measured under the accelerated conditions (25±2° C./60±5% RH) as in Test Example 1 are shown in Table 8.

TABLE 8

| | RP-HPLC (Remaining rate, %) | | | | | SE-HPLC (Remaining rate, %) | | | | |
|---|---|---|---|---|---|---|---|---|---|---|
| | 0 W | 1 W | 2 W | 3 W | 4 W | 0 W | 1 W | 2 W | 3 W | 4 W |
| Preparation | 100 | 99.9 | 98.4 | 96.8 | 94.0 | 100 | 99.7 | 99.1 | 98.0 | 97.0 |
| Example 38 | | | | | | | | | | |

### 9. Test of Liquid Formulation of Preparation Example 39

The remaining rates of the formulation of Preparation Example 39 measured under the accelerated conditions (25±2° C./60±5% RH) as in Test Example 1 are shown in Table 9.

TABLE 9

| | RP-HPLC (Remaining rate, %) | | | | | SE-HPLC (Remaining rate, %) | | | | |
|---|---|---|---|---|---|---|---|---|---|---|
| | 0 W | 1 W | 2 W | 3 W | 4 W | 0 W | 1 W | 2 W | 3 W | 4 W |
| Preparation | 100 | 100 | 98.5 | 97.0 | 94.0 | 100 | 99.7 | 99.0 | 98.0 | 97.0 |
| Example 39 | | | | | | | | | | |

### 10. Test of Liquid Formulation of Preparation Example 40

The osmolarity, conductivity, viscosity, maximum gliding force, and remaining rate of the formulation were measured in the same manner as in Test Example 1.

As a result, the formulation of Preparation Example 40 had an osmolarity of 59.3 mOsm/kg, a conductivity of 3.45 mS/cm, and a viscosity value of 1.26 cP at room temperature (20 to 25° C.). The formulation of Preparation Example 40 had a maximum gliding force of 2.471 N (at 4.725 mm/s velocity with a 29-G syringe), and 1.834 N (at 2.835 mm/s velocity with a 29-G syringe).

The remaining rates of the formulation of Preparation Example 10 under the accelerated conditions (25±2° C./60±5% RH) are shown in Table 10.

TABLE 10

| | RP-HPLC (Remaining rate, %) | | | | | SE-HPLC (Remaining rate, %) | | | | |
|---|---|---|---|---|---|---|---|---|---|---|
| | 0 W | 1 W | 2 W | 3 W | 4 W | 0 W | 1 W | 2 W | 3 W | 4 W |
| Preparation | 100 | 99.8 | 99.4 | 98.8 | 98.6 | 100 | 100 | 99.7 | 99.5 | 99.4 |
| Example 40 | | | | | | | | | | |

### 11. Test of Liquid Formulation of Preparation Example 41

The osmolarity, conductivity, viscosity, maximum gliding force, and remaining rate of the formulation were measured in the same manner as in Test Example 1.

As a result, the formulation of Preparation Example 41 had an osmolarity of 1721.7 mOsm/kg, a conductivity of 31.20 mS/cm, and a viscosity value of 2.02 cP at room temperature (20 to 25° C.). The formulation of Preparation Example 41 had a maximum gliding force of 2.952 N (at 4.725 mm/s velocity with a 29-G syringe), and 1.922 N (at 2.835 mm/s velocity with a 29-G syringe).

The remaining rates of the formulation of Preparation Example 41 under the accelerated conditions (25±2° C./60±5% RH) are shown in Table 11.

TABLE 11

| | RP-HPLC (Remaining rate, %) | | | | | SE-HPLC (Remaining rate, %) | | | | |
|---|---|---|---|---|---|---|---|---|---|---|
| | 0 W | 1 W | 2 W | 3 W | 4 W | 0 W | 1 W | 2 W | 3 W | 4 W |
| Preparation | 100 | 99.7 | 99.4 | 98.8 | 98.9 | 100 | 100 | 99.2 | 99.1 | 99.0 |
| Example 41 | | | | | | | | | | |

### 12. Test of Liquid Formulation of Preparation Example 42

The osmolarity, conductivity, viscosity, maximum gliding force, and remaining rate of the formulation were measured in the same manner as in Test Example 1.

As a result, the formulation of Preparation Example 42 had an osmolarity of 1964.3 mOsm/kg, a conductivity of 10.56 mS/cm, and a viscosity value of 2.66 cP at room temperature (20 to 25° C.). The formulation of Preparation Example 42 had a maximum gliding force of 7.482 N (at 4.725 mm/s velocity with a 29-G syringe), and 5.688 N (at 2.835 mm/s velocity with a 29-G syringe).

The remaining rates of the formulation of Preparation Example 42 under the accelerated conditions (25±2° C./60±5% RH) are shown in Table 12.

TABLE 12

| | RP-HPLC (Remaining rate, %) | | | | | SE-HPLC (Remaining rate, %) | | | | |
|---|---|---|---|---|---|---|---|---|---|---|
| | 0 W | 1 W | 2 W | 3 W | 4 W | 0 W | 1 W | 2 W | 3 W | 4 W |
| Preparation | 100 | 99.7 | 99.4 | 99.0 | 98.9 | 100 | 100 | 99.3 | 99.1 | 98.7 |
| Example 42 | | | | | | | | | | |

### 13. Test of Liquid Formulation of Preparation Example 43

The osmolarity, conductivity, viscosity, maximum gliding force, and remaining rate of the formulation were measured in the same manner as in Test Example 1.

As a result, the formulation of Preparation Example 43 had an osmolarity of 316.3 mOsm/kg, a conductivity of 13.38 mS/cm, and a viscosity value of 64.9 cP at room temperature (20 to 25° C.). The formulation of Preparation Example 43 had a maximum gliding force of 7.482 N (at 4.725 mm/s velocity with a 29-G syringe), and 5.688 N (at 2.835 mm/s velocity with 29-G syringe).

The remaining rates of the formulation of Preparation Example 43 under the accelerated conditions (25±2° C./60±5% RH) are shown in Table 13.

TABLE 13

| | RP-HPLC (Remaining rate, %) | | | | | SE-HPLC (Remaining rate, %) | | | | |
|---|---|---|---|---|---|---|---|---|---|---|
| | 0 W | 1 W | 2 W | 3 W | 4 W | 0 W | 1 W | 2 W | 3 W | 4 W |
| Preparation | 100 | 99.3 | 96.5 | 92.9 | 87.8 | 100 | 98.5 | 94.3 | 87.0 | 69.1 |
| Example 43 | | | | | | | | | | |

### 14. Patient-Friendly (PF) Index Evaluation

To determine whether the prepared liquid formulations satisfy the conditions of Equation 1 as above, the PF index of each formulation was calculated. The results are shown in Table 14.

TABLE 14

| Preparation Example | PF index |
|---|---|
| Preparation Example 1 | 8.549 |
| Preparation Example 2 | 8.718 |
| Preparation Example 3 | 9.352 |
| Preparation Example 4 | 9.506 |
| Preparation Example 5 | 3.010 |
| Preparation Example 6 | 4.632 |
| Preparation Example 40 | 2.427 |
| Preparation Example 41 | 19.139 |
| Preparation Example 42 | 22.085 |
| Preparation Example 43 | 8.851 |

As shown in Tables 1 to 13, in the cases of Preparation Examples 37-39 including a high-concentration polysorbate-based non-ionic surfactant, some data indicated a remaining rate problem. In addition, the liquid formulation of Preparation Example 43 showed a serious remaining rate problem.

Referring to Table 14, the liquid formulation of Preparation Example 43 was found to have an osmolarity within an appropriate range, but still a high maximum gliding force, and thus may still cause pain to patients. The liquid formulation of Preparation Example 41 was found to have a maximum gliding force within an appropriate range, but a high osmolarity, and may still cause pain to patients. Meanwhile, the liquid formulations according to the embodiments were found to have an appropriate osmolarity, a maximum gliding force of 5 N or less, and an appropriate PF index of 3 to 10. Therefore, it was found that, in the case of the liquid formulation according to an embodiment having a PF index of 3 to 10, wherein osmolarity and maximum gliding force are major factors in determining the PF index, the preferred liquid formulation may be administered to patients without causing pain.

### Test Example 2. Evaluation of Remaining Rate of Liquid Formulation According to Concentration of Polysorbate-Based Non-Ionic Surfactant

In the same manner as in Test Example 1, after a 4-week storage test under accelerated conditions (25±2° C./60±5% RH), the remaining rates of eflapegrastim were measured using the liquid formulations of Preparation Examples 1 and 44-46.

### 1. Test of Liquid Formulation of Preparation Example 1

The remaining rates under accelerated conditions of the liquid formulation of Preparation Example 1 are shown in Table 15.

TABLE 15

| | RP-HPLC (Remaining rate, %) | | | | | SE-HPLC (Remaining rate, %) | | | | |
|---|---|---|---|---|---|---|---|---|---|---|
| | 0 W | 1 W | 2 W | 3 W | 4 W | 0 W | 1 W | 2 W | 3 W | 4 W |
| Preparation | 100 | 99.7 | 99.7 | 99.7 | 99.3 | 100 | 100 | 99.9 | 99.7 | 99.6 |
| Example 1 | | | | | | | | | | |

### 2. Test of Liquid Formulation of Preparation Example 44

The remaining rates under accelerated conditions of the liquid formulation of Preparation Example 44 are shown in Table 16.

TABLE 16

| | RP-HPLC (Remaining rate, %) | | | | | SE-HPLC (Remaining rate, %) | | | | |
|---|---|---|---|---|---|---|---|---|---|---|
| | 0 W | 1 W | 2 W | 3 W | 4 W | 0 W | 1 W | 2 W | 3 W | 4 W |
| Preparation | 100 | 99.4 | 99.4 | 99.2 | 98.5 | 100 | 99.9 | 99.3 | 98.9 | 98.5 |
| Example 44 | | | | | | | | | | |

### 3. Test of Liquid Formulation of Preparation Example 45

The remaining rates under accelerated conditions of the liquid formulation of Preparation Example 45 are shown in Table 17.

TABLE 17

| | RP-HPLC (Remaining rate, %) | | | | | SE-HPLC (Remaining rate, %) | | | | |
|---|---|---|---|---|---|---|---|---|---|---|
| | 0 W | 1 W | 2 W | 3 W | 4 W | 0 W | 1 W | 2 W | 3 W | 4 W |
| Preparation | 100 | 99.0 | 98.5 | 98.1 | 97.4 | 100 | 99.8 | 99.0 | 98.3 | 97.5 |
| Example 45 | | | | | | | | | | |

### 4. Test of Liquid Formulation of Preparation Example 46

The remaining rates under accelerated conditions of the liquid formulation of Preparation Example 46 are shown in Table 18.

TABLE 18

| | RP-HPLC (Remaining rate, %) | | | | | SE-HPLC (Remaining rate, %) | | | | |
|---|---|---|---|---|---|---|---|---|---|---|
| | 0 W | 1 W | 2 W | 3 W | 4 W | 0 W | 1 W | 2 W | 3 W | 4 W |
| Preparation | 100 | 98.8 | 98.1 | 97.2 | 96.5 | 100 | 99.6 | 98.5 | 97.5 | 96.7 |
| Example 46 | | | | | | | | | | |

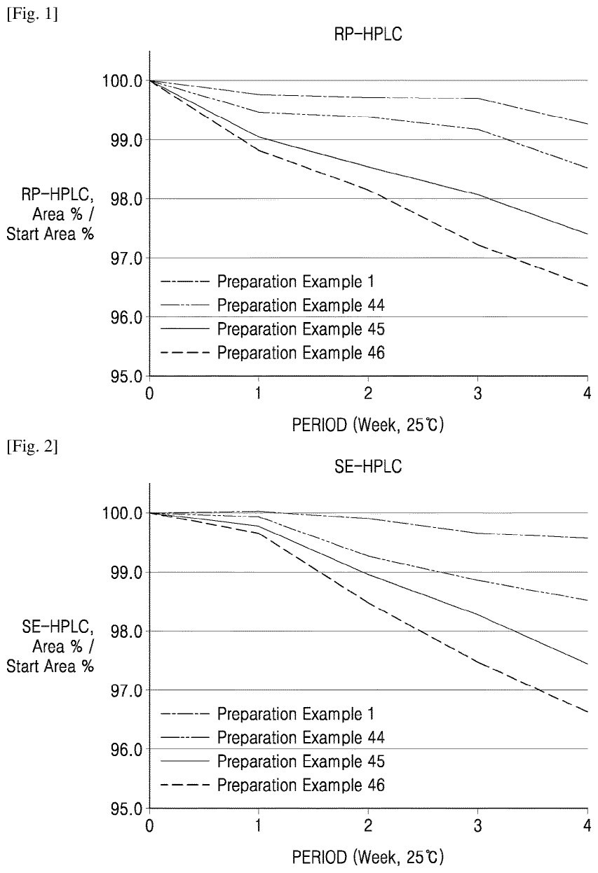

FIGS. 1 and 2 show results confirming changes in remaining rate of eflapegrastim according to concentration of the surfactant in the liquid formulations according to embodiments. As shown in FIGS. 1 and 2, the concentration of the surfactant showed a high correlation with the remaining rate of the liquid formulations according to embodiments, and these experimental results indicate that the concentration of surfactant is a main factor affecting the remaining rate of a liquid formulation including a high-concentration of eflapegrastim. From the above results, it was found that the liquid formulation according to any of the embodiments is different from existing liquid formulations in terms of the amount of active ingredient and the preparation method, and accordingly may have high formulation stability (for example, remaining rate) and high patient-friendly (PF) index, and thus may be administered without causing pain to patients.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

1. A liquid eflapegrastim formulation comprising eflapegrastim and a buffer material, wherein
a concentration of the eflapegrastim is about 11 mg/mL to about 66 mg/mL;
a patient-friendly (PF) index of the liquid formulation, represented by Equation 1, is 10 or less;
[Equation 1]
Patient-friendly (PF) index=Osm (mOsm/kg)/100+MGF (N) wherein, in Equation 1, Osm indicates the osmolarity value of the liquid formulation, and MGF indicates a value of maximum gliding force when the liquid formulation is injected with a 29-gauge (29 G) syringe at a rate of 2.835 mm/s;
an osmolarity of the liquid formulation is about 100 mOsm/kg to about 800 mOsm/kg;
a maximum gliding force (MGF) of the liquid formulation is 5 N or less when injected with a 29-gauge (29 G) syringe at a rate of about 2.835 mm/s, or 7N or less at a rate of about 4.725 mm/s; and
a remaining rate of eflapegrastim after storage at a temperature of 23° C. to 27° C. and a relative humidity of about 55% to 65% is 95% or greater, as measured by reversed phase high-performance liquid chromatography (RP-HPLC) or size-exclusion high-performance liquid chromatography (SE-HPLC).

2. The liquid eflapegrastim formulation of claim 1, wherein the liquid formulation has a conductivity of 15 mS/cm or less.

3. The liquid eflapegrastim formulation of claim 1, wherein the remaining rate of eflapegrastim is 98% or greater.

4. The liquid eflapegrastim formulation of claim 1, wherein the liquid formulation has a viscosity of 4 cP or less at a room temperature of 20° C. to 25° C.

5. The liquid eflapegrastim formulation of claim 1, wherein a concentration of the buffer material is about 5 mM to about 100 mM.

6. The liquid eflapegrastim formulation of claim 5, wherein the buffer material is citric acid and/or citrate.

7. The liquid eflapegrastim formulation of claim 1, further comprising a stabilizing agent.

8. The liquid eflapegrastim formulation of claim 7, wherein the stabilizing agent comprises mannitol.

9. The liquid eflapegrastim formulation of claim 8, wherein a concentration of the mannitol is about 1% to about 20% (w/v) of the liquid formulation.

10. The liquid eflapegrastim formulation of claim 1, further comprising a surfactant.

11. The liquid eflapegrastim formulation of claim 10, wherein the surfactant is a poly sorbate-based non-ionic surfactant.

12. The liquid eflapegrastim formulation of claim 11, wherein the polysorbate-based non-ionic surfactant is selected from the group consisting of Polysorbate 20, Polysorbate 40, Polysorbate 60, and Polysorbate 80.

13. The liquid eflapegrastim formulation of claim 12, wherein a final concentration of the polysorbate-based non-ionic surfactant after the liquid formulation is concentrated is about 0.0001% to about 0.5% (w/v) of the total liquid formulation.

14. The liquid eflapegrastim formulation of claim 1, wherein the liquid formulation has a pH of about 4 to about 8.

15. The liquid eflapegrastim formulation of claim 1, further comprising a tonicity modifier.

16. The liquid eflapegrastim formulation of claim 15, wherein the tonicity modifier is sodium chloride.

17. The liquid eflapegrastim formulation of claim 15, wherein a concentration of the tonicity modifier is about 5 mM to about 200 mM.

18. The liquid eflapegrastim formulation of claim 1, wherein the liquid formulation is pre-treated using a purification column.

19. The liquid eflapegrastim formulation of claim 18, wherein the pre-treated liquid formulation is concentrated after buffer exchange with a buffer which does not contain a polysorbate-based non-ionic surfactant.

20. A liquid eflapegrastim formulation comprising eflapegrastim, a buffer material, and a surfactant, wherein
a concentration of the eflapegrastim is about 11 mg/mL to about 66 mg/mL;
a concentration of the buffer material is about 5 mM to about 100 mM; and
a concentration of the surfactant after the liquid formulation is concentrated is about 0.001% to about 5% (w/v) of the total liquid formulation, and a concentration of the surfactant after the liquid formulation is concentrated is about 0.001% to about 5% (w/v) of the total liquid formulation.

21. The liquid eflapegrastim formulation of claim 20, wherein the surfactant is a poly sorbate-based non-ionic surfactant.

22. The liquid eflapegrastim formulation of claim 20, wherein the liquid formulation comprises:
about 11 mg/mL to about 66 mg/mL of the eflapegrastim;
about 5 mM to about 100 mM of citric acid and/or citrate; and
about 0.001% to about 5% (w/v) of a polysorbate-based non-ionic surfactant.

23. The liquid eflapegrastim formulation of claim 21, wherein the polysorbate-based non-ionic surfactant is selected from the group consisting of Polysorbate 20, Polysorbate 40, Polysorbate 60, and Polysorbate 80.

24. The liquid eflapegrastim formulation of claim 21, wherein the liquid formulation comprises:
about 11 mg/mL to about 66 mg/mL of the eflapegrastim;
about 5 mM to about 100 mM of sodium citrate;
about 0.001% to about 0.5% (w/v) of Polysorbate 80;
about 1% to about 20% (w/v) of mannitol; and
about 5 mM to about 200 mM of sodium chloride.

25. The liquid eflapegrastim formulation of claim 20, wherein the osmolarity of the liquid formulation is about 100 mOsm/kg to about 800 mOsm/kg.

26. The liquid eflapegrastim formulation of claim 20, wherein the liquid formulation has a conductivity of 15 mS/cm or less.

27. A method of preventing, alleviating, or treating neutropenia in a patient having compromised white blood cell production comprising administering to the patient a therapeutically effective amount of the liquid eflapegrastim formulation of claim 1.

28. The method of claim 27, wherein the neutropenia is severe chronic neutropenia or febrile neutropenia.

29. The method of claim 27, wherein the liquid eflapegrastim formulation is administered after the patient is treated with adjuvant or neoadjuvant chemotherapy.

30. The method of claim 29, wherein the liquid eflapegrastim formulation is administered between 1 and 5 days after the patient is treated with adjuvant or neoadjuvant chemotherapy.

31. The method of claim 30, wherein the adjuvant or neoadjuvant chemotherapy is a combination of docetaxel and cyclophosphamide.

32. The method of claim 30, wherein a second dose of the liquid eflapegrastim formulation is administered between 15 and 25 days after a first dose of the liquid eflapegrastim formulation is administered to the patient.

33. The method of claim 30, wherein the therapeutically effective amount is a unit dosage form selected from: 25 ug/kg, 50 ug/kg, 100 ug/kg, and 200 ug/kg.

34. The method of claim 30, wherein the therapeutically effective amount is 13.2 mg of the liquid eflapegrastim formulation in a 0.6 mL dosage volume.

35. The method of claim 30, further comprising administering to the patient a therapeutically effective amount of a second agent.

36. The claim of claim 35, wherein the second agent is an anti-cancer agent.

37. The method of claim 29, wherein the liquid eflapegrastim formulation is administered to the patient within about 6 hours, about 5 hours, about 2 hours, about 1 hour of completion of chemotherapy.
